(19) United States
(12) Patent Application Publication
(10) Pub. No.: US 2023/0014433 A1
(43) Pub. Date: Jan. 19, 2023

(54) SECURING AND DELIVERING GRAFTS FOR ENDOTHELIAL KERATOPLASTY

(71) Applicant: Lions Eye Institute for Transplant and Research, Inc., Tampa, FL (US)

(72) Inventors: Eric Abdullayev, Tarpon Springs, FL (US); Arthur C. Kurz, Safety Harbor, FL (US)

(73) Assignee: Lions Eye Institute for Transplant and Research, Inc., Tampa, FL (US)

(21) Appl. No.: 17/902,371

(22) Filed: Sep. 2, 2022

Related U.S. Application Data
(63) Continuation-in-part of application No. 16/727,217, filed on Dec. 26, 2019.
(60) Provisional application No. 62/785,368, filed on Dec. 27, 2018.
(60) Provisional application No. 62/785,430, filed on Dec. 27, 2018.
(60) Provisional application No. 63/240,144, filed on Sep. 2, 2021.

(51) Int. Cl. A61F 2/14; A61F 2/00

(52) U.S. Cl. CPC A61F 2/148; A61F 2/142; A61F 2/0095

(57) ABSTRACT

Ophthalmic tissue delivery devices that include an injector and injector carriers and methods for endothelial keratoplasty surgical procedures are provided. Injectors include a conduit sized to accommodate ophthalmic tissue. One end of the injector is configured for insertion into a patient's eye to inject ophthalmic tissue. Injector carriers include a container, a cap configured to seal an opening of the container. The injector can be coupled to a pressure actuated valve and a syringe where activating the syringe opens the value and allows fluid to flow from the syringe, through the valve, and through the injector to dispense ophthalmic tissue from the injector into a patient's eye without the need to physically contact the ophthalmic tissue.

### CROSS-REFERENCE TO RELATED APPLICATIONS

This application is a continuation-in-part of U.S. Nonprovisional patent application Ser. No. 16/727,217 filed on Dec. 26, 2019, which itself claims priority to U.S. Provisional Application No. 62/785,368 filed on Dec. 27, 2018 and U.S. Provisional Application No. 62/785,430 filed on Dec. 27, 2018. This application further claims priority to U.S. Provisional Application No. 63/240,144 filed on Sep. 2, 2021. The entire disclosures of the above applications are incorporated herein by reference.

### TECHNICAL FIELD AND BACKGROUND

The present technology relates to devices and methods for introducing donor ophthalmic tissue into an anterior chamber of an eye and for the storage and transportation of ophthalmic tissue. More particularly, the technology relates to fluid injection delivery devices that deliver an ophthalmic tissue graft to a patient's eye without the need to touch the tissue graft during transplant, as well as carriers and methods of using the same for the storage, transportation, and transplantation of Descemet's membrane and endothelium into a recipient's cornea.

Ophthalmic tissue transplantation can be dramatically improved using endothelial keratoplasty (“EK”) surgical techniques. Such surgical techniques include: (i) Descemet's Stripping Automated Endothelial Keratoplasty (“DSAEK”) where an approximately 50-100 micron layer of the thin cornea stroma, Descemet's membrane (“DM”) with a layer of endothelial cells can be removed from a donor cornea and transplanted into a recipient patient's eye; (ii) Descemet's Membrane Endothelial Keratoplasty (“DMEK”) where an approximately 10 micron layer of the Descemet's membrane with a layer of endothelial cells can be removed from a donor cornea and transplanted into a patient recipient's eye; and (iii) Pre-Descemet's Endothelial Keratoplasty (“PDEK”) where the pre-Descemet's layer (“PDL”) along with Descemet's membrane and a layer of endothelial cells is transplanted.

Despite the improvements realized using endothelial keratoplasty surgical techniques, delivery of ophthalmic tissue, including the fragile and thin layer of the endothelial cells from the donor cornea, can be a difficult process. The donor tissue must be carefully stored and transported in a aseptic manner to the surgical site to mitigate the risk of damaging the tissue. Extraction of ophthalmic tissue from a tissue donor can occur at various time periods prior to the transplantation procedure, so the ophthalmic tissue may need to be stored in a preservation medium for a significant time period spanning days. Prepared donor tissue can be stored with a layer of endothelium inward or outward. The ophthalmic tissue, which can be quite fragile, may also need to be transported from a location where the tissue was extracted from a donor to another location where the transplantation procedure is to occur, and the tissue is to be transplanted into a recipient's anterior chamber and then positioned there in correct orientation—endothelial layer inward.

The ophthalmic tissue storage and transplant devices must allow surgeons to carefully extract the tissue for transplant to the recipient patient without damaging the single layer of endothelial cells that are critical for restoring healthy vision. The corneal endothelium serves the important function of pumping fluid out of the corneal stroma to prevent the occurrence of edematous haze, which results in cloudy vision. Notably, the corneal endothelium does not regenerate following damage or loss of cells, so preventing damage to the endothelium during transplant is critical for successful transplant surgery.

To facilitate the transplantation, a tissue delivery device or introducer, such as an injector, can be preloaded with the ophthalmic tissue with endothelial layers inward or outward, and the delivery device is used to insert the ophthalmic tissue into the eye. Conventional surgical techniques require surgeons to load tissue into the injector delivery device at the time of surgery just prior to dispensing tissue into the patient's eye. Pre-loading the donor tissue into the delivery device prior to surgery reduces the risk of errors experienced when preparing the tissue and loading tissue into a delivery device intraoperatively. Errors experienced intraoperatively may cause cancellation of the surgical procedure.

Additionally, DSAEK, DMEK, and PDEK procedures require that an incision be made in the patient's eye prior to introducing the donor graft into the patient's anterior chamber using fluid via the delivery device. By providing an fluid injection delivery device preloaded with the ophthalmic tissue, the time investment in the transplantation procedure can be minimized while improving clinical outcomes.

The size of an incision must be large enough to accommodate the delivery device. But at the same time, the incision should also be minimized to promote patient safety during surgery, healing, and recovery time and to prevent serious conditions such as acute ophthalmic fluid loss and chronic astigmatism caused by irregularities in the surface of the cornea. During surgery, ophthalmic fluid may escape from the incision resulting in the collapse of the patient's cornea, which may prevent the transplanted tissue from becoming properly positioned in a patient's anterior chamber. Further, sutures used to close the incision following surgery can apply tension to the surrounding cornea tissue resulting in surface irregularities and scarring that can cause long-term negative impacts to patient vision.

Transplantation of donor ophthalmic tissue into a recipient's eye consequently faces several challenges, including aspects related to storage of the ophthalmic tissue, transportation of the ophthalmic tissue, and ultimately the transplantation procedure itself especially positioning donor tissue in correct anatomical orientation (i.e., with the endothelial layer inward). There is a need to provide improved devices and methods for careful, safe, expedient, and successful storage, transport, and introduction of ophthalmic tissue into an eye, including optimization of DSAEK, DMEK, and PDEK procedures and preparation. It is, thus, an object of the present invention to provide devices and methods that allow donor ophthalmic tissue to be pre-loaded into a delivery device that can be used to store, transport, and introduce the tissue into a patient's anterior chamber while minimizing damage to the tissue, enhancing patient safety, improving clinical outcomes, reducing surgeon stress, reducing intraoperative donor tissue preparation errors, and minimizing length of the surgical procedure.

### SUMMARY

The present technology includes articles of manufacture, systems, and processes that relate to storage, transport, and introducing ophthalmic tissue, including Descemet's membrane and endothelium into a recipient patient's eye for various restorative procedures, including DSAEK, DMEK, and PDEK. Further areas of applicability will become apparent from the description provided herein. The description and specific examples are intended for purposes of illustration only and are not intended to limit the scope of the present disclosure.

In one embodiment, an ophthalmic tissue delivery apparatus for performing endothelial keratoplasty includes an injector formed as an elongated hollow body. The injector has an inner surface that defines a conduit sized and configured to safely hold ophthalmic tissue for storage and transport to a surgical site. The injector has a beveled first end with a first opening, and a second end with a second opening. The injector is coupled to a pressure actuated valve. In one embodiment, the pressure activated value includes a first portion configured for coupling to a syringe, a valve main body, a deformable stopper disposed in the main body, and a second portion in fluid communication with the injector second end.

In one example embodiment, the conduit can be formed with an inner diameter between 1.1 millimeters and 3.2 millimeters, and the injector elongated body has an outer diameter between 2.2 millimeters and 4 millimeters. The beveled first end can have a bevel outer diameter that less is than 4 millimeters to minimize incision size during surgery. The bevel end can include a leading portion and a trailing portion.

In another embodiment, the pressure actuated value is connected to the injector through a resilient member. The resilient member is elongated with a channel extending from a first aperture to a second aperture. The resilient member is coupled to the second portion of the pressure-actuated valve by extending the second portion partially through the resilient member first aperture. The resilient member is coupled to the injector second end by extending the injector second end partially through the resilient member second aperture. The elongated hollow body of the injector can be formed with a bulb disposed on the outer surface of the elongated hollow body. The resilient member fits around the bulb to secure the injector to the resilient member and place the injector in fluid communication with the pressure actuated valve through the resilient member.

The ophthalmic tissue delivery apparatus can be connected to a syringe filled with a balanced salt solution that is coupled to the first portion of the pressure actuated value. Thus, fluid flows from the syringe, through the valve, through the resilient member, and through the injector to apply pressure to ophthalmic tissue in the injector to dispense the ophthalmic tissue graft from the beveled end of the injector into a patient's eye. The ophthalmic tissue graft is dispensed along with fluid from the injector and the syringe to help maintain pressure within the patient's eye to prevent corneal collapse during surgery. The ophthalmic tissue graft is dispensed in an orientation such that the endothelial layer is facing down such that the tissue graft becomes properly oriented within the anterior chamber of the patient's eye who is receiving the donor tissue graft.

To facilitate storage and transport of ophthalmic tissue grafts after extraction from a donor, some embodiments include an injector carrier. The injector carrier includes a container having an opening and a cap configured to seal the opening of the container. At least a portion of the injector is disposed within the container. The conduit and the container are each at least partially filled with a corneal storage medium.

An ophthalmic tissue graft is stored within the conduit in a tri-fold configuration to prevent damage to the tissue and facilitate dispensing the tissue during surgery. That is, the tri-fold configuration helps the tissue naturally unfold into the proper curvature and position in the patient's eye. The conduit houses an ophthalmic tissue graft that has an endothelium layer and a stromal layer. The ophthalmic tissue graft is in a folded configuration with the endothelium layer facing inward and the stromal layer facing outward to contact the conduit inner surface. The ophthalmic tissue graft relaxes slightly and is not folded too tight such that the endothelium layer can contact the corneal storage medium while within the conduit so as to provide proper irrigation to the tissue.

A method for performing endothelial keratoplasty for an eye of a patient is disclosed that includes the step of loading the conduit with a corneal storage medium. The conduit is also loaded with an ophthalmic tissue graft having an endothelium layer and a stromal layer opposite the endothelium layer. The ophthalmic tissue graft is folded with the endothelium layer facing inward and the stromal layer facing outward to contact the conduit inner surface. A syringe is coupled to the first portion of the pressure actuated valve. The syringe has a barrel containing a fluid and a plunger that is partially housed within the barrel. Depressing the plunger applies pressure to the fluid to dispense the fluid through the value.

An incision is created in the eye of a patient where the incision is less than 4 millimeters in width, which is the smallest incision known or disclosed in trade literature. The beveled first end of the injector is inserted into the eye of a patient through the incision. The beveled first end occupies substantially the entire incision to seal the incision and mitigate fluid loss from the eye. The bevel is inserted into the eye such that the bevel faces upward in a direction away from the interior of the patient's eye. Depressing the plunger dispenses the ophthalmic tissue graft from the conduit through the beveled first end into the eye of the patient.

### BRIEF DESCRIPTION OF THE DRAWINGS

The drawings described herein are for illustrative purposes only of selected embodiments and not all possible implementations, and are not intended to limit the scope of the present disclosure.

FIG. 1 shows an embodiment of a needle injector for performing surgical procedures introducing ophthalmic tissue grafts into an eye.

FIG. 2 shows an embodiment of a needle injector for performing surgical procedures introducing ophthalmic tissue grafts into an eye with a layer of endothelium cells facing outward.

FIG. 3 shows an assembled perspective view of an embodiment of a carrier for a needle injector for performing surgical procedures introducing ophthalmic tissue grafts into an eye with a layer of endothelium cells facing outward.

FIG. 4 shows a partially disassembled perspective view of the embodiment of a carrier for a needle injector accordingly to FIG. 3.

FIG. 5 shows an exploded perspective view of the embodiment of a carrier for a needle injector according to FIG. 3.

FIG. 6 shows cross-sectional assembled view of the embodiment of a carrier for a needle injector according to FIG. 3.

FIG. 7 shows a needle portion of a needle injector being introduced into an eye for delivery of a graft.

FIG. 8 shows the wound profile of the needle injector of FIG. 7.

FIG. 9 shows a scalpel making an incision in an eye so that an injector without a needle portion can introduce a graft through the incision.

FIG. 10 shows the wound profile of the scalpel of FIG. 9.

FIG. 11A shows an embodiment of an injector delivery device for performing no-touch surgical procedures introducing ophthalmic tissue grafts into an eye with a layer of endothelium cells facing inward.

FIG. 11B shows an embodiment of an injector delivery device for performing no-touch surgical procedures introducing ophthalmic tissue grafts into an eye with a layer of endothelium cells facing inward.

FIG. 12 illustrates use of an injector delivery device for performing no-touch surgical procedures introducing ophthalmic tissue grafts into an eye.

FIG. 13 shows an exploded view of an injector delivery device, resilient member, and valve for performing no-touch surgical procedures introducing ophthalmic tissue grafts into an eye.

FIG. 14 shows an assembled view of an injector delivery device, resilient member, valve, and syringe for performing no-touch surgical procedures introducing ophthalmic tissue grafts into an eye.

FIG. 15 shows an assembled view of a carrier for an injector delivery device for performing no-touch surgical procedures introducing ophthalmic tissue grafts into an eye.

FIG. 16A is a side view of an example embodiment of an injector delivery device illustrating dimensions of the delivery.

FIG. 16B is a top view of an example embodiment of an injector delivery device illustrating dimensions of the delivery.

FIG. 16C is a first end view of an example embodiment of an injector delivery device illustrating dimensions of the delivery.

FIG. 17 illustrates the results of a study examining tissue damages resulting from forceps push DSAEK surgical techniques that introduce ophthalmic tissue grafts into an eye.

FIG. 18 illustrates the results of a study examining tissue damage resulting from DSAEK surgical techniques including glide pull-through technique, forceps push technique as compared to no-touch fluid injection technique of introduction of ophthalmic tissue grafts with endothelium inwards into an eye.

### DETAILED DESCRIPTION

The following description of the inventive technology disclosed herein provides examples of the subject matter, devices, methods of making, and methods of using one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

### Technology Applications

The present technology improves administration of ophthalmic tissue into an eye to perform endothelial keratoplasty, including Descemet's Stripping Automated Endothelial Keratoplasty, Descemet's membrane endothelial keratoplasty, and Pre-Descemet's endothelial keratoplasty. For these surgical techniques, at least a portion of the Descemet's membrane, and endothelium cells, is removed from a donor cornea and transplanted into a patient/recipient's eye. The layer of the Descemet's membrane with a layer of endothelial cells can be inserted into the front portion (e.g., anterior chamber) of the patient's eye using particular fluid injector or delivery device. In one embodiment well suited for DMEK procedures, the fluid injector delivery device is a needle injector that can be used to cut the patient's eye so that there is no need to pre-cut or make an incision in the eye prior to use of the needle injector. The graft can then be dispensed from the needle injector into the eye of the patient. In another embodiment that is particularly well suited for use in DSAEK procedures, the injector delivery can be inserted into small, pre-cut incisions in a patient's eye for delivery of the ophthalmic tissue using fluid without the need to mechanically contact the tissue using instrumentation/forceps.

### Needle Injector Embodiments

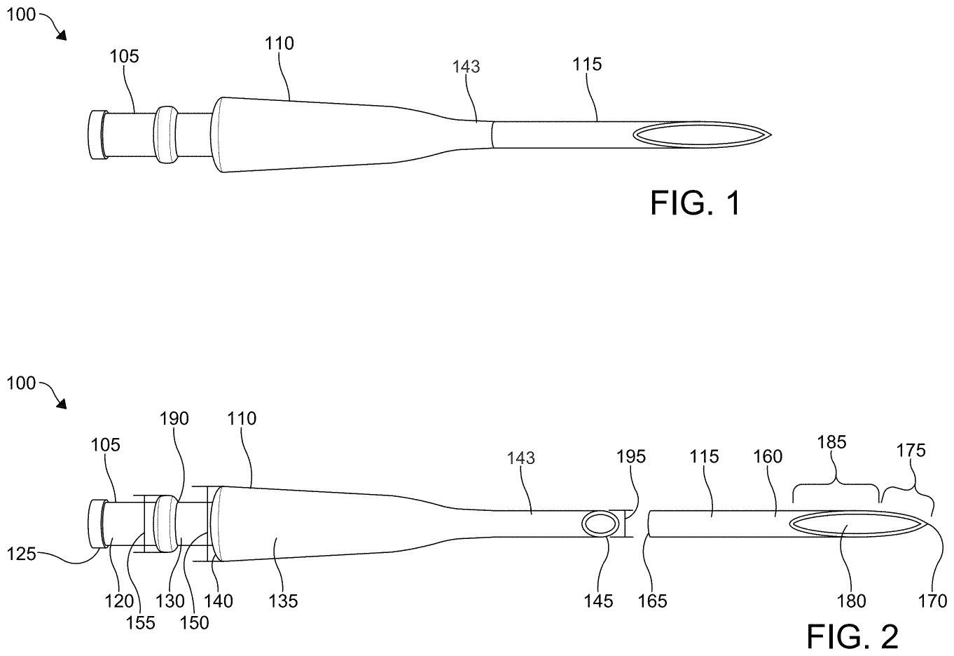

With reference to FIGS. 1-2, an embodiment of a needle-type injector delivery device 100 (“needle injector”) is shown having a first portion 105, a second portion 110, and a third portion 115. The first portion 105 includes a first conduit 120 having a first end 125 and a second end 130. The second portion 110 includes a second conduit 135 having a first end 140, a delivery segment 143, and a beveled second end 145 proximal to the delivery segment 143. The second conduit 135 has a maximum diameter 150 greater than a maximum diameter 155 of the first conduit 120. The first end 140 of the second conduit 135 is fluidly coupled to the second end 130 of the first conduit 120. A bulb 190 is disposed between the first end 125 and the second end 130 of the first conduit 120. The bulb 190 can be used to engage or define a coupling with other components, such as an elastomeric resilient member 225. The bulb 190 can also be used to engage or define coupling with fluid manipulation devices, such as a syringe having a barrel and plunger.

At least a portion of the second conduit 135 tapers in a direction from the first end 140 of the second conduit 135 towards the delivery segment 143 and the second end 145 of the second conduit 135. The third portion 115 can be optionally used during transplant surgery where the third portion 115 has a second end 170 with a cutting surface 175 used to create an incision in a patient's eye for dispensing a tissue graft. Alternatively, the third portion 115 can be removed, and surgery is conducted with an ophthalmic tissue graft dispensed from the second conduit 135 through the beveled dispensing segment 143 and into a patient's eye. The second end 145 of the second conduit 135 has a diameter 195 that is less than the maximum diameter 155 of the first conduit 120. The second end 145 and the delivery segment 143 of the second conduit 135 are also configured to fit into the first end 165 of the third conduit 160 for the third portion 115 when the third portion is used during surgery to create a small incision in the patient's eye for delivering tissue into the anterior chamber.

The third portion 115 includes a third conduit 160 having a first end 165 and a second end 170. The first end 165 of the third conduit 160 is configured to be reversibly and fluidly coupled to the second end 145 of the second conduit 135. The second end 170 of the third conduit 160 is configured with a cutting surface 175 for cutting and penetrating eye tissue. The cutting surface 175 can be configured as a lancet style blade. The cutting surface 175 is located at a distal end of an opening 180 at the second end 170 of the third conduit 160. The remainder of the opening 180 has a non-cutting surface 185.

The first end 165 of the third conduit can be reversibly coupled to the second end 145 of the second conduit 135 so that the needle injector 100 can be used or manipulated without the third portion 115 attached. This affords the user the option of performing an endothelial keratoplasty procedure where an incision is made in the eye and the second end 145 of the second conduit 135 is inserted through the incision to dispense ophthalmic tissue into the eye. Alternatively, the user has the option of having the first end 165 of the third conduit fluidly coupled to the second end 145 of the second conduit 135, where the second end 170 of the third conduit 160 is inserted into the eye so that the cutting surface 175 cuts and penetrates the eye. The ophthalmic tissue is then dispensed from the second conduit 135 of the needle injector 100 through the third conduit 160 of the needle injector 100 into the eye of the patient.

The second conduit 135 can form a chamber that is sized and shaped to accommodate, store, and dispense a graft of ophthalmic tissue for endothelial keratoplasty. For example, the ophthalmic tissue can be loaded into the needle injector 100 through the first conduit 120 and stored within the chamber of the second conduit 135. At least a portion of the second conduit 135 can taper in a direction from the first end 140 of the second conduit towards the second end 145 of the second conduit 135. Where the second conduit 135 includes the chamber, the chamber can be tapered in this fashion. The taper can facilitate dispensing the ophthalmic tissue from the second conduit 135 into the eye. The second end 145 of the second conduit 135 can have a diameter that is less than the maximum diameter of the first conduit 120. In this way, it can be easier to load the ophthalmic tissue into the needle injector 100 through the larger maximum diameter of the first conduit 120, while the smaller diameter of the second end 145 of the second conduit 135 minimizes the size of the portion of the needle injector 100 that is introduced into the eye. The second end 145 of the second conduit 135 can also be configured to fit into the first end 165 of the third conduit 160. In this way, the ophthalmic tissue does not have to conform to a smaller diameter or overcome a lip or edge in the transition between the second conduit 135 and the third conduit 160.

The first conduit 120 can be about 0.8 cm to about 0.9 cm in length and can provide entry for loading the prepared ophthalmic tissue/graft (e.g., DMEK or PDEK graft) and can be configured for connection to a syringe. Where the first conduit 120 includes the bulb 190, the bulb 190 can provide a tighter connection with a coupling means of an injector carrier 200. The second conduit 135 can be about 1.9 cm to about 2.0 cm in length and can have a diameter to provide adequate corneal storage media irrigation to ophthalmic tissue contained therein. For example, where the second conduit 135 includes a chamber or a tapered chamber, the chamber can provide a volume optimized for contact between the ophthalmic tissue and cornel storage medium. That is, the chamber is sized to avoid the circumstance where the ophthalmic tissue becomes folded or constrained in a manner that does not allow the corneal storage medium to contact the ophthalmic tissue, which could result in damage to the tissue. The ophthalmic tissue can be positioned in the second conduit 135 during transportation/storage. The third conduit 160 can be about 1.9 cm to about 2.0 cm in length and can provide the exit for the ophthalmic tissue. The second end 170 of the third conduit 160 can be inserted by puncture into a recipient's anterior chamber where the ophthalmic tissue is then unloaded. The total length of the needle injector can be about 4.5 cm to about 4.8 cm.

One end of the needle injector 100 can be configured with a cutting surface 175 for cutting and penetrating eye tissue. For example, the second end 170 of the third conduit 160 can be configured like a portion of a hypodermic needle having a hollow portion or conduit 160 ending in a sharp point defined by a cutting surface 175 that is intended to puncture through outer eye tissue; e.g., cutting through the cornea to enter the anterior chamber of the eye. The cutting surface 175 can include a beveled edge of a length and shape that can be tailored to optimize cutting dependent on the desired incision size. The cutting surface 175 can be formed of a material that can be sharpened or manufactured with a sharp edge configured for cutting through the outer surface of the eye allowing the needle injector 100 to penetrate into the interior of the eye. For example, the third portion 115 of the needle injector 100 can be configured much in the same fashion as the end of a beveled hypodermic needle, where a leading portion at the distal end of the beveled opening can form a cutting surface 175 or lancet and the trailing portion of the beveled opening can form a non-cutting surface 185. In this way, the amount of the eye and subsequent wound profile is minimized, where the remainder of the beveled opening can push through the incision without having to increase the size of the incision opening.

The needle injector 100 can be manufactured and assembled in various ways. In some embodiments, the needle injector 100 can be formed as a single integrated unit including all three portions connected to each other as one piece, where no assembly is necessary prior to use. In other embodiments, the needle injector can initially include two separate parts—a first part including the first and second portions 105 & 110 and a second part including the third portion 115. The first part can be formed of a single piece of glass that embodies the first portion 105 and the second portion 110. The second part can be formed of a single piece of metal that embodies the third portion 115. For full assembly of the needle injector 100, the first and second parts can be coupled by disposing the second end 145 of the second conduit 135 into the first end 165 of the third conduit 160. Coupling can be reversible or can be made essentially permanent by press-fitting or through use of an adhesive, for example.

Alternatively, the third portion 115 of the needle injector 100 can be detachable during use of the needle injector 100. This provides the user the option of using the needle injector 100 with the third portion 115 to cut through the outer surface of the eye to allow the needle injector 100 to penetrate into the interior of the eye and dispense ophthalmic tissue therefrom. Alternatively, a user has the option of using the needle injector 100 by detaching and removing the third portion 115, where the second end 145 of the second portion 110 of the needle injector 100 can be inserted through a pre-cut incision in the eye. The ophthalmic tissue graft is then dispensed from the second conduit 135, through the delivery segment 143, and through the second end 145, through a pre-cut incision and into patient's eye. For example, the delivery segment 143 and second end 145 of the second portion 110 of the needle injector 100 can be dimensioned to fit into an incision used in prior endothelial keratoplasty methods.

The first, second, and third conduits can be made from glass or plastic. Use of plastic has the advantage of being less susceptible to breakage and easier and less expensive to manufacture using blow molding, injection molding, or extrusion techniques. In another embodiment, the cutting surface 175 of the second end 170 of the third conduit 160 can be comprised of glass or metal. For example, the third conduit 160 can be a hollow metal tube having a beveled end similar to a hypodermic needle. Use of the beveled edge has the advantage of facilitating insertion of the second end 170 of the third portion 115 through an incision in the cornea and enhancing the efficacy of the cutting surface 175.

### Injector Carrier Embodiment

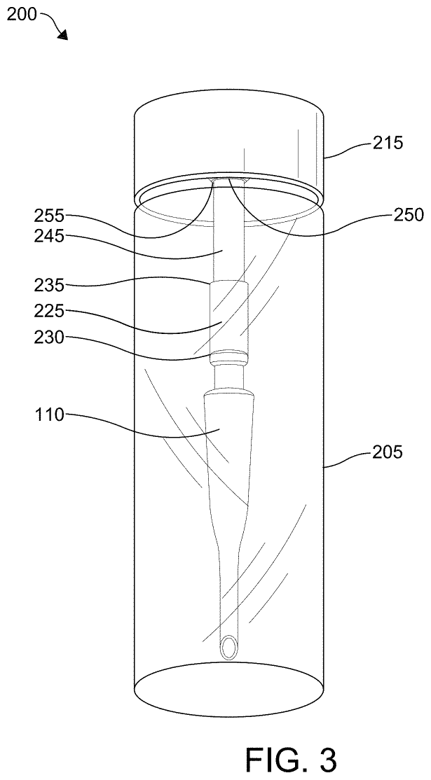

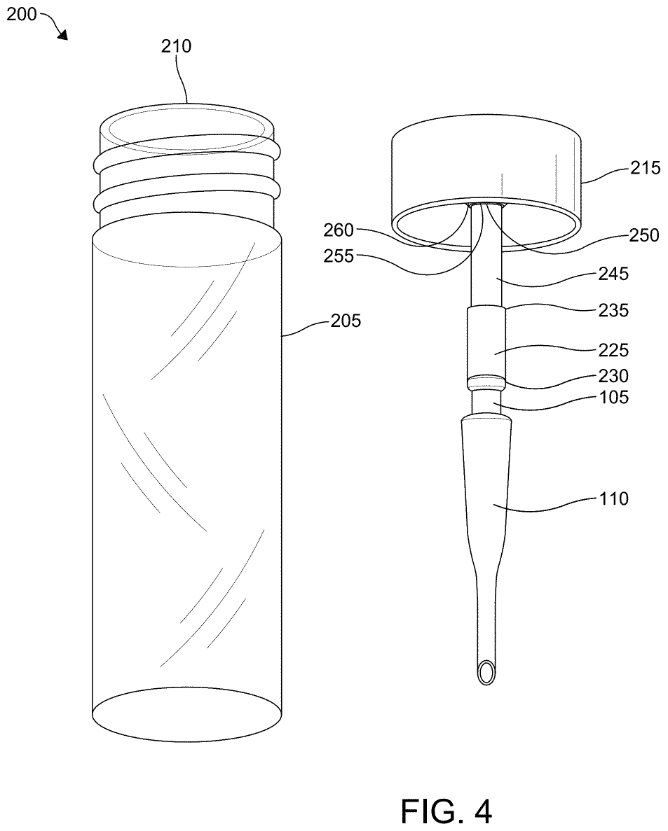

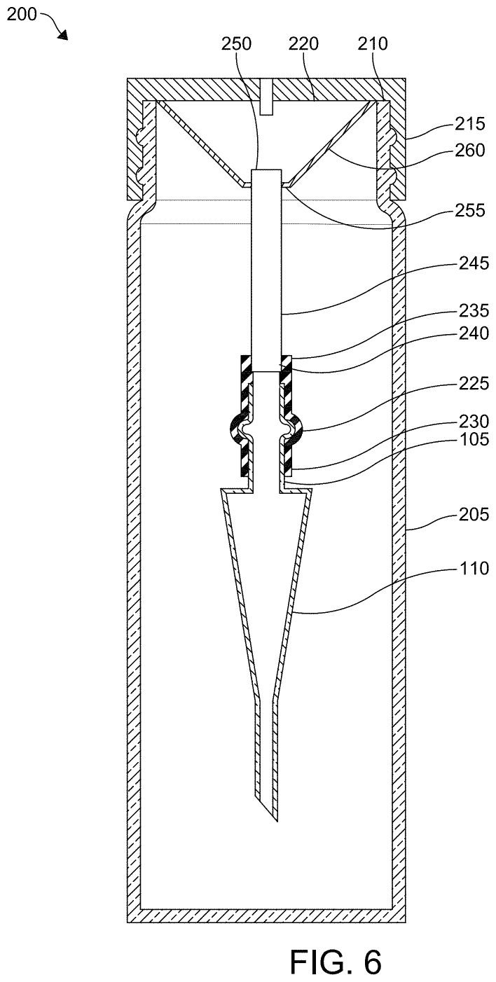

The needle injector 100 can be used in conjunction with an injector carrier 200. With reference to FIGS. 3-6, an embodiment of an injector carrier 200 is shown in conjunction with the first portion 105 and second portion 110 of the needle injector 100 of FIGS. 1-2. It should be recognized that while the injector carrier 200 in use with the needle injector 100 of FIGS. 1-2, the injector carrier 200 can be used with other injectors or introducers used in the art for dispensing ophthalmic tissue in an endothelial keratoplasty procedure. It should be further recognized that injector carrier 200 can be configured to accommodate just the first and second portions 105 & 110 of the needle injector 100 as shown (e.g., the third portion 115 is uncoupled from the second portion 110) or where the third portion 115 is coupled to the second portion 110.

Where the injector carrier 200 is used in conjunction with just the first and second portions 105 & 110 of the needle injector 100, the third portion 115 can be provided or packaged separately (e.g., packaged similarly to a sterile hypodermic needle). The embodiment of the injector carrier 200 depicted includes a container 205 having an opening 210, a cap 215 configured to seal the opening 210 of the container 205, and a coupling means 220 configured to couple the first portion 105 of the needle injector 100 to the cap 215 and allow at least the first portion 105 and the second portion 110 of the needle injector 100 to be disposed within the container 205 when the cap 215 seals the opening 210 of the container 205. As shown in FIGS. 3 and 6, at least the first and second portions 105 & 110 of the needle injector 100 can be disposed within the container 205 without contacting the container 205.

The container 205 is sized so that the needle injector 100 holds a vertical, not horizontal, position when disposed in the container 205. This is significant as conventional devices store the ophthalmic tissue in a horizontal position, which damages the endothelium layer because the endothelial layer settles by gravity onto the surface of the delivery device causing endothelial cell damage.

In the embodiment shown, the coupling means 220 of the injector carrier 200 includes a resilient member 225 having a first end 230 configured to fit over the first end 125 of the first conduit 120 of the needle injector 100. The resilient member 225 also has a second end 235 that fits over a first end 240 of a stem 245 coupled to the cap 215. The second end 250 of the stem 245 is fit into an opening 255 within a sealing member 260 coupled to the cap 215, where the sealing member 260 is configured to seal the opening 210 of the container 205. The sealing member 260 is has a frustoconical shape where the larger end engages the cap 215 and the smaller end has the opening 255 that receives the second end 250 of the stem 245. The resilient member 225 can be configured as a short piece of elastic plastic tubing or collar serving to couple the needle injector 100 (e.g., the first end 125 of the first conduit 120) to the stem 245 depending from the sealing member 260 and cap 215.

The injector carrier 200 is used to transport and protect the needle injector 100, including where the needle injector 100 holds ophthalmic tissue in contact with corneal storage medium within the container 205. The container 205 of the injector carrier 200 can include corneal storage medium, including an amount of corneal storage medium sufficient to contact ophthalmic tissue in the needle injector 100 when at least the first portion 105 and the second portion 110 of the needle injector 100 are disposed within the container 205 when the cap 215 seals the opening 210 of the container 205.

The injector carrier 200 can be sized to accommodate the needle injector 100 where the first and second portions 105 & 110 are separated from the third portion 115. The container 205 of the injector carrier 200 can also be sized to accommodate the needle injector 100 where first and second portions 105 & 110 are coupled to the third portion 115 (e.g., where the first end 165 of the third conduit 160 is fluidly coupled to the second end 145 of the second conduit 135). In any instance, the injector carrier 200 can be configured to hold the needle injector 100 within the container 205 without contacting an interior wall or bottom of the container 205.

### Working Examples of DMEK and PDEK Surgical Procedures

The following examples were used to evaluate endothelial cell viability of prepared DMEK grafts of ophthalmic tissue in conjunction with use of the needle injector 100. All grafts of ophthalmic tissue were pre-loaded into a needle injector 100 for use in DMEK and PDEK. The ophthalmic tissue was injected into an anterior chamber, replicating the anterior chamber of the human eye, as follows. Three (n=3) DMEK grafts were prepared using a no-touch hydrodissection method and stained with Trypan Blue, then trephined with an 8 mm donor punch. Each graft was loaded into a needle injector 100, stored for 1 day in Optisol-GS at 2-8° C., and then injected into the anterior chamber replicating the anterior chamber of the human eye.

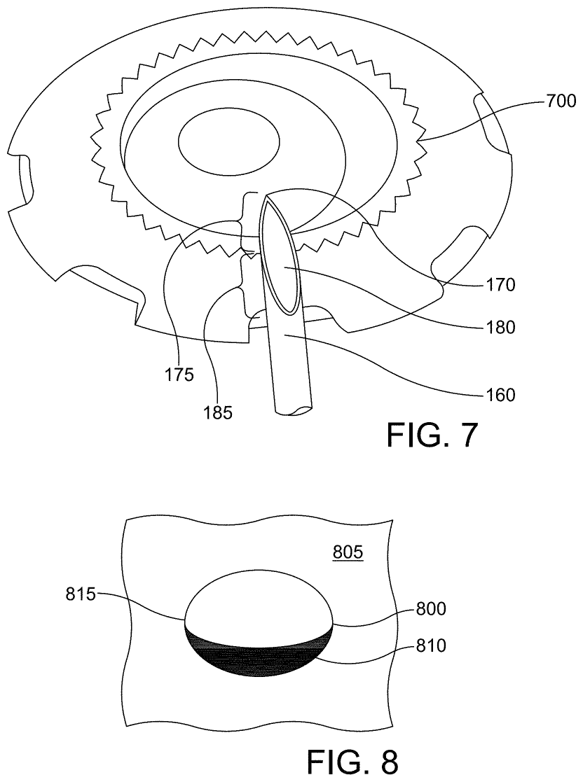

FIG. 7 depicts insertion of the second end 170 of the third conduit 160 into the anterior chamber 700, wherein the cutting surface 175 cuts and penetrates into the anterior chamber 700. The ophthalmic tissue is then dispensed from the second conduit 135 of the needle injector 100 through the third conduit 160 of the needle injector 100 into the anterior chamber 700.

A close-up of an insertion profile 800 into the outer surface 805 of the anterior chamber 700 is shown in FIG. 8. A cut 810 is made by the cutting surface 175. A mark 815 is visible where the non-cutting surface 185 pushes through into the anterior chamber 700, but does not cut the outer surface 805, as the entire opening 180 at the second end 170 of the third conduit 160 is inserted into the anterior chamber 700. As can be seen from the insertion profile 800, the actual cut 810 into the outer surface 805 of the anterior chamber 700 is less than the outer diameter of the third conduit 160 (e.g., a cut of about 1.3 mm for a 1.6 mm outer diameter) which minimizes trauma to the eye tissue and improves healing. For example, the non-cutting surface 185 of the beveled opening 180 can push through the cut 810 without further cutting and increasing the size of the cut 810 in the outer surface 805 of the anterior chamber 700.

Viability of the corneal endothelium introduced into the anterior chamber 700 was evaluated by slit lamp, specular microscopy, and cell staining. Cells were stained with Trypan Blue (0.4%), processed, and analyzed using “ImageJ” software to quantitate the stained portion. In the needle injector 100, the prepared graft of ophthalmic tissue moves in one direction. That is, the graft is loaded from the wider end at the first end 125 of the first conduit 120 (e.g., about 2 mm inner diameter) in order to reduce the potential for endothelial damage. The graft of ophthalmic tissue then is dispensed and unloads from the narrow end of glass tube (e.g., first and second portions 105, 110), through the second end 145 of the second conduit 135, which is inside the first end 165 of the third conduit 160 (e.g., the needle). The first end 165 of the third conduit 160 is placed over the narrow second end 145 of the second conduit 135. The second end 145 of the second conduit 135 can be positioned inside of the third conduit 160 proximal to the beginning of the beveled opening 180 to reduce exposure of the ophthalmic tissue to the metal of the third conduit 160. In this configuration, contact between the endothelium and metal inner surface of the third conduit 160 is eliminated.

Endothelial cell density was evaluated as follows. Prior to DMEK tissue preparation, mean endothelial cells density was determined to be about 2434 cells/mm². After the graft of ophthalmic tissue for DMEK was prepared, mean endothelial cell density was determined to be about 2634 cells/mm². After dispensing through the needle injector 100 into the anterior chamber 700, mean endothelial cell density was determined to be about 2486 cells/mm². Endothelial cell staining evaluation confirmed viable endothelium with only about a 3% increase of damaged cells (e.g., from 1.5% to 4.5%). Thus, the present needle injector 100 can be used to provide efficient dispensing of ophthalmic tissue into an eye while minimizing damage to the introduced graft.

The following example, compares the wound architecture as illustrated by the insertion profile 800 of the needle injector 100 with the wound architecture resulting from use of a glass injector as used in other methods of performing endothelial keratoplasty. The outside diameter of the third conduit 160 (e.g., the metal needle) of the needle injector 100 used was 1.6 mm. As such, the needle injector 100 and the glass injector as used in other methods of performing endothelial keratoplasty each required the same sized diameter to be inserted into the eye. As shown in FIG. 8, the needle injector 100 results in an insertion profile 800 where the actual cut 810 into the outer surface 805 of the anterior chamber 700 is less than the outer diameter of the third conduit 160 (e.g., a cut of about 1.3 mm for a 1.6 mm outer diameter).

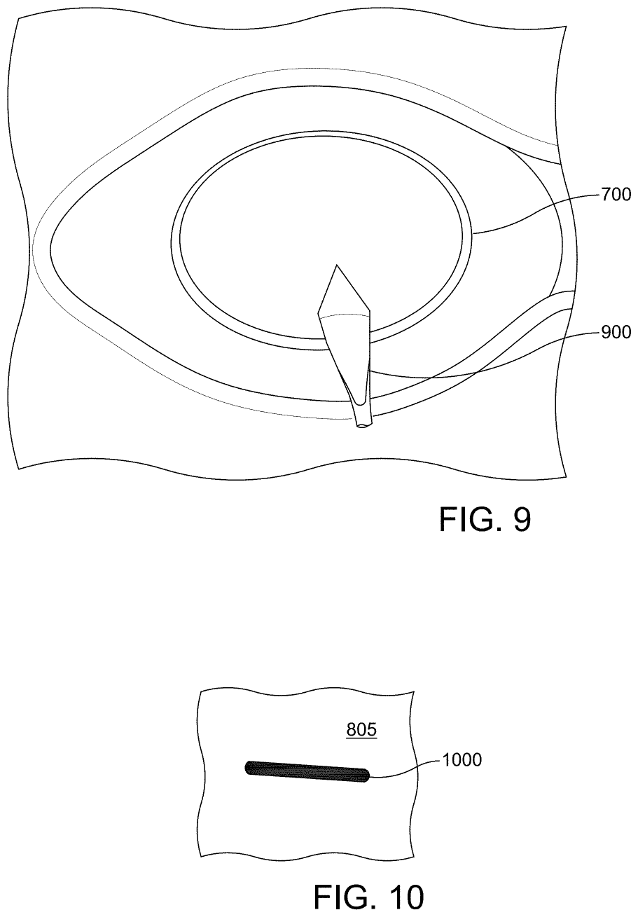

Insertion of the end of the glass injector used in other methods of performing endothelial keratoplasty, however, requires that an incision be first made with a scalpel 900, as shown in FIG. 9. The scalpel 900 is removed and the dispensing end of the glass injector is then inserted into the anterior chamber 700. This results in the insertion profile 1000 shown in FIG. 10. The insertion profile 1000 in this instance is equal to the actual cut into the outer surface 805 of the anterior chamber 700, which is greater than the diameter of the dispensing end of the glass injector. In particular, it is not possible to insert the glass injector through a 1.6 mm incision, even though the dispensing end of the glass injector has a 1.6 mm diameter. Fully inserting the glass injector requires an incremental additional incision size of at least 0.4 to 0.5 mm up to a minimum incision size of 2.2 mm. When a 2.2 mm blade of the scalpel 900 enters into tissue, a 2.2 mm straight wound is formed, as shown in FIG. 10.

Multiple measurements have identified that use of a glass needle injector requires about a 0.6 mm incremental wound size than the present needle injector 100, where the needle injector results in a cut 810 of about 1.6 mm versus the glass needle injector incision size of about 2.2 mm. Other types of glass injectors and introducers used in the art can even require incisions from about 2.6 mm to about 3.6 mm. Accordingly, the different wound architecture of using the needle injector 100 and the smaller incision/cut formed in the eye surface serve to minimize trauma and improve healing of the eye. Likewise, wound architecture of the crescent shape of the insertion profile 800 from the needle injector 100 versus a blade incision promotes sutureless wound healing. These aspects can combine to be less invasive and result in smaller wounds, where the ability to forgo the use of a suture can further reduce potential for astigmatism. Thus, the present needle injector 100 has demonstrated certain benefits and advantages over glass injectors or introducers that require a scalpel incision for their use.

### DSAEK Injector Embodiment Adapted for No-Touch Surgical Applications

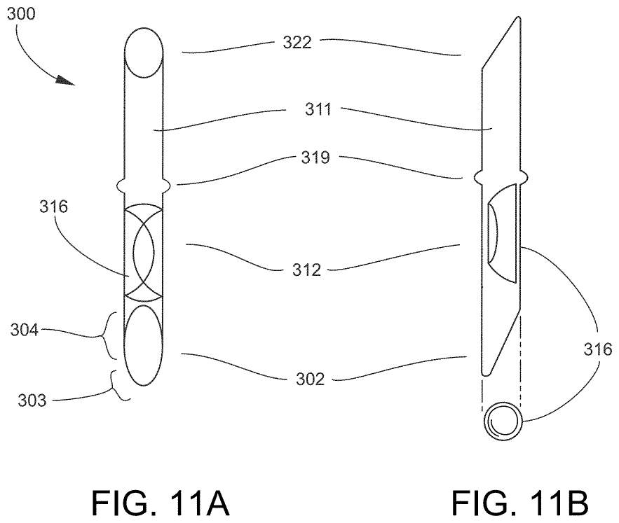

An embodiment of an injector delivery device 300 is shown in FIGS. 11A-11B that is particularly adapted for use in no-touch surgical procedures, including specifically DSAEK procedures. The delivery device 300 embodiment for DSAEK procedures is adapted to minimize ophthalmic tissue damage, ease the transplant procedure while minimizing the incision size, reduce endothelial cell damage, promote patient health and safety and improve clinical outcomes. Skilled artisans will appreciate that the below-described devices and techniques can be adapted for use in endothelial keratoplasty surgical procedures generally, including DSAEK, DMEK, and PDEK procedures. However, the devices and techniques have particular advantages when applied to DSAEK surgical techniques, which have previously not been suitable for no-touch fluid injection procedures during transplant surgery

Tissue implanted during DSAEK surgical procedures is thicker than tissue implanted through other surgical techniques, such as DMEK. As a result, all previously known methods for performing DSAEK surgical procedures rely on physical or mechanical contact with the tissue to be transplanted. However, contact with the cornea tissue results in damage to the delicate endothelium portion of the tissue, which is a single cell layer thick. The corneal endothelium tissue is critical for clear vision, but the endothelium does not regenerate following cell damage or loss. Consequently, avoiding harm to the corneal endothelium is an important factor for successful surgery. Further, known DSAEK surgical techniques require incision sizes of 4 mm to 5 mm that can cause patient harm and delay surgical recovery and cause post-operative astigmatism.

Conventional DSAEK surgical techniques include the use of different devices for folded, pull-through and push techniques. These existing techniques rely on physically or mechanically contacting the ophthalmic tissue and inserting the tissue through an incision in the patient's cornea. In case of a folded surgical technique, surgeons use forceps to insert the folded tissue graft through an incision in the patient's cornea. Push techniques use a special device with a moving plunger to “push” or insert the ophthalmic tissue graft through the incision into the patient's anterior chamber. The ophthalmic tissue is folded prior to insertion and naturally unfolds after being inserted through the incision. As the tissue unfolds, it takes the convex shape of the patient's cornea.

Pull-through techniques require the surgeon to load tissue into a platform such as a glide, and then with use of a forceps, pull the ophthalmic tissue through a corneal incision into and across the anterior chamber. For the pull through technique, at least two incisions are created in a patient's eye. Unloading forceps are inserted through one incision and brought across the anterior chamber to a second “primary” incision. The unloading forceps are used to grasp the ophthalmic tissue from platform that is placed just outside the primary incision. The forceps then pull the tissue into the anterior chamber. For folded, push, and pull-through techniques, the transplanted ophthalmic tissue is mechanically manipulated during transplant resulting in the loss of endothelium cells that do not regenerate.

Conventional DSAEK surgical techniques also require larger incision sizes of 4 mm to 5 mm that can negatively impact patient health and safety. During surgery, larger incision sizes result in ocular fluid escaping from the incision. Loss of fluid can lead to collapse of the patient's cornea, which prevents the transplanted tissue from properly unfolding after insertion through an incision and cause increased endothelial cell loss. To prevent cornea collapse, the patient's cornea is inflated during surgery by injecting balanced salt solution (“BSS”) into the patient's eye through an anterior chamber maintainer cannula that is separate from the injector device.

After transplant, larger incision sizes may require additional sutures that apply tension to the surface of the patient's cornea. This tension causes surface irregularities that lead to astigmatism that negatively impacts a patient's vision following surgery. By contrast, smaller incision sizes have benefits that include preventing cornea collapse during surgery, less endothelial cell loss, expediting patient recovery, and minimizing the deleterious effects on a patient's vision resulting from astigmatism or other conditions.

The negative effects caused by mechanically manipulating the cornea tissue and large incision sizes are mitigated using the fluid injector delivery device 300, or simply “injector,” that is shown in FIGS. 11A-11B through FIG. 15. The injector 300 is formed as an elongated hollow body 312 that defines a conduit 311 for housing ophthalmic tissue 316. The inner sidewall of the injector 300 that forms the conduit 311 is a smooth surface so that the conduit 311 extends substantially along the length of the injector delivery device 300. Thus, the inner sidewall is without interruption or contours that might contact and damage an ophthalmic tissue graft housed inside of the conduit 311.

The injector delivery device 300 can be cylindrical with first end or “injection end” 302 that has a first opening and a second end or “coupling end” 322 that has a second opening. The injector delivery device 300 optionally includes a bulb 319 formed on the exterior surface of the injector delivery device 300. The tissue is loaded into the coupling end 322 before being output from the injection end 302 during surgery. The optional bulb 319 is formed between the first end/injection end 302 and the second end/coupling end 322. The bulb 319 serves to engage or define an enhanced coupling with an elastic resilient member 225 or a fluid manipulation device, such as a syringe having a barrel and plunger.

The injection end 302 optionally includes a cutting surface 303 and a non-cutting surface 304 such that the injector delivery device 300 functions as a needle injector. In embodiments using a cutting surface 303, the cutting surface 303 can be configured as a lancet-style blade so that the injector delivery device 300 can penetrate a patient's cornea to dispense ophthalmic tissue without the need for a separate scalpel or other cutting instrument. The injection end 302 is beveled to optimize the efficacy of the cutting surface 303 (when present) and to minimize damage to the incision during insertion of the injector delivery device 300 into the anterior chamber That is, the injection end 302 can be configured so that the narrow leading portion enters the incision first without damaging the incision, and the beveled trailing portion follows without tearing or otherwise damage the tissue as the injector delivery device 300 is inserted into the anterior chamber. In this manner, the wound profile of the incision is minimized, where the remainder of the beveled opening can push through the incision without having to increase the size of the incision opening.

The injector delivery device 300 is formed with a smaller outer diameter than existing devices used for DSAEK surgical procedures, which may be a conventional or modified Jones tube and made of glass, plastic, or other material. In part because of the smaller outer diameter and the beveled features of the injection end 302, the injector delivery device 300 shown in the attached figures can be used to successfully perform no-touch DSAEK surgical procedures with incision sizes of approximately 3.2 mm. The smaller incision sizes may allow for sutureless wound healing following surgery, which results in faster recovery time and mitigation of risks from conditions such as astigmatism.

The smaller incision size in turn enables closed surgical procedures that cannot be achieved using conventional DSAEK surgical techniques. For closed procedures, the injection end 302 of the injector delivery device 300 is placed into patient's anterior chamber and occupies most of incision opening while ophthalmic tissue is dispensed into the anterior chamber using fluid injection. The injector delivery device 300 thereby creates a seal that minimizes the volume of escaping ocular fluid to prevent cornea collapse. The procedure is also more safe as the ophthalmic tissue is not exposed to the ambient environment and does not contact other instruments such as forceps.

The interior conduit 311 defines a chamber that is sized to house an ophthalmic tissue graft 316 from a tissue donor while providing adequate corneal storage media irrigation. That is, the conduit 311 has a minimum allowable diameter to avoid the circumstance where the ophthalmic tissue graft 316 becomes folded or constrained in a manner that does not allow the corneal storage and preservation medium to contact the endothelium of the ophthalmic tissue graft 316, which could result in damage to the tissue/endothelium 316. Typical ophthalmic tissue diameter sizes used for grafts may be 7.5 mm to 8.75 mm in diameter, and the tissue is tri-folded with the layer of endothelial cells facing inward to permit loading into the injector delivery device conduit 311 for storage.

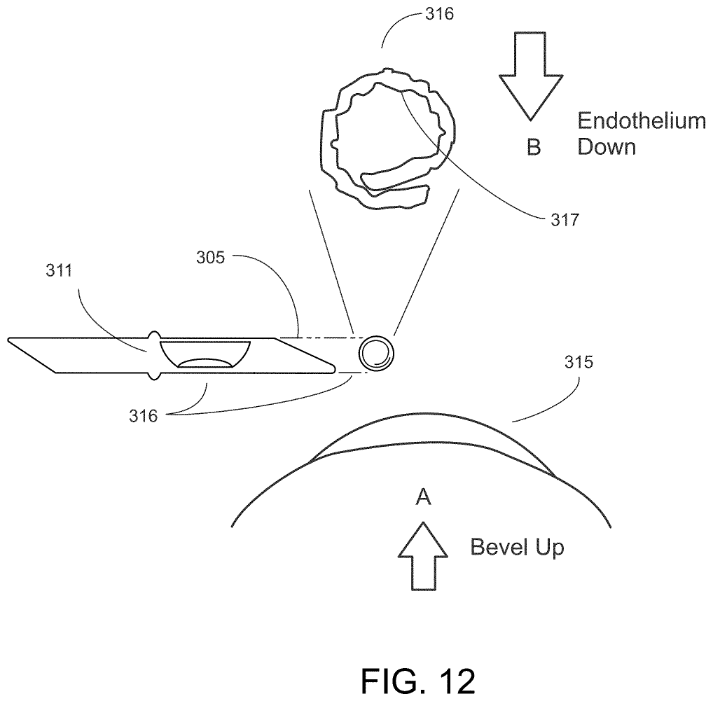

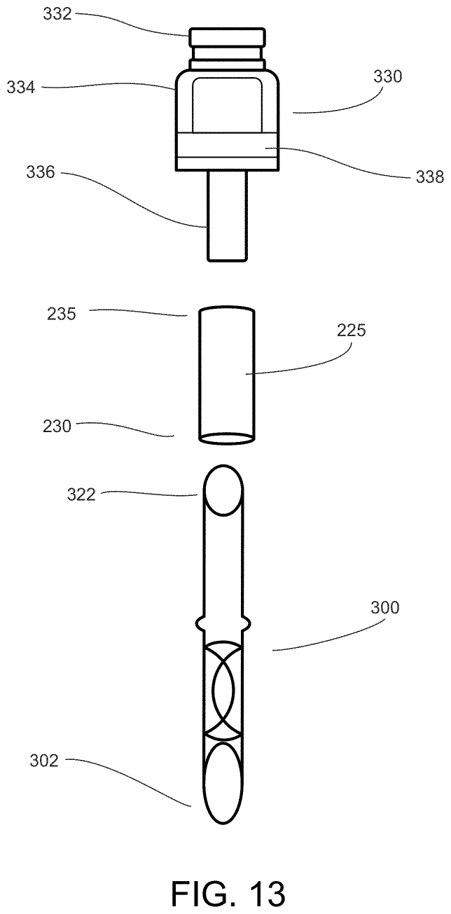

The chamber defined by the interior conduit 311 is sized to allow the ophthalmic tissue graft 316 to be tri-folded as shown in FIG. 12. The ophthalmic tissue graft 316 is tri-folded with the endothelium layer 317 folded inward so that it does not contact the surface of the conduit 311. The stromal side of the ophthalmic tissue graft 316 faces outward and naturally engages and pushes against sidewalls of the conduit 311 as the tissue relaxes while housed in the conduit 311. The force created by the ophthalmic tissue graft 316 pushing against the inner sidewall of the conduit 311 holds the tissue graft 316 in place and does not allow the tissue graft 316 to rotate or change orientation within the conduit 311. Put another way, the tri-folded ophthalmic tissue graft 316 naturally moves toward an unfolded state when not housed within the conduit 311. Thus, the conduit 311 is sized with a maximum diameter such that once the ophthalmic tissue graft 316 is housed within the conduit 311, the ophthalmic tissue graft 316 does rotate in the conduit 311 or move in a manner that might damage the tissue graft 316.

Thus, the conduit 311 should not be too small and should have a minimum diameter that allows irrigation to the ophthalmic tissue graft 316. But the conduit 311 maximum diameter should not be too large such that the ophthalmic tissue graft 316 is not secured within the conduit and/or that allows the ophthalmic tissue graft 316 to rotate and change orientation.

The configuration and orientation does not change during storage and transport and protects the delicate endothelium layer 317 during storage, transport, and transplant of the ophthalmic tissue graft 316. The orientation of the ophthalmic tissue 316 folds relative to the orientation of the bevel 305 in the injection end 302 of the injector delivery device 300 can play a role in successful transplant by facilitating the proper unfolding and orientation of the ophthalmic tissue after it is injected into the anterior chamber. The injector delivery device 300 shown in the attached figures allows the ophthalmic tissue folds to maintain their orientation during storage and transport, thereby enhancing the chances of a successful transplant surgery. As shown in FIG. 12, the two ends of the ophthalmic tissue graft 316 are oriented toward the “long” side of the injection end 302 bevel. The ophthalmic tissue graft 316 should not rotate from the shown orientation within the conduit 311 or else risk having the ophthalmic tissue graft 316 unfold with the incorrect orientation when disposed within a patient's anterior chamber.

With reference to FIG. 12, during a DSAEK surgical procedure, the beveled surface 305 of the injector delivery device injection end 322 is pointed upward in the “A” direction while the injector delivery device 300 penetrates a patient's cornea 315. This allows the ophthalmic tissue 316 to be injected with the endothelium layer 317 pointed downward in the “B” direction. This technique is referred to as “bevel-up, endo-down” and allows the ophthalmic tissue 316 to naturally unfold and take the shape of a patient's cornea 315 after the tissue 316 is dispensed into the anterior chamber of the cornea in proper orientation 315.

As illustrated by the example embodiment dimensions, the coupling end 322 of the injector delivery device 300 can optionally be formed with a larger diameter than the injection end 302. Following extraction of ophthalmic tissue from a donor, the tissue is placed into the symmetrical tri-fold configuration using forceps and loaded into the injector delivery device 300 through the larger diameter coupling end 322. Both the injection end 302 and the coupling end 322 are formed with a bevel 305 and the injection end is used to inject ophthalmic tissue into a patient's eye through an incision (i.e., the coupling end 322 becomes the injection end 302 with a larger diameter).

The injector delivery device 300 can be made from a glass material, such as 7740 Pyrex® borosilicate glass. In other embodiments, the injector delivery device 300 can be made from a plastic material that is less susceptible to breaking and less expensive to manufacture using blow molding, injection molding, or extrusion. In yet another embodiment, when a cutting surface 303 is present at the injection end 302, the cutting surface 303 can be made from a metal material suitable for penetrating and cutting ophthalmic tissue.

Turning to FIGS. 13-15, the injector delivery device 300 is shown with other components that facilitate storage, transportation, and transplant of ophthalmic tissue. The coupling end 322 of the injector delivery device 300 can be coupled to a first aperture at a first end 230 of a flexible, elongated resilient member 225. The resilient member 225 second aperture at a second end 235 is in turn coupled to the second, male or injection portion 336 of a pressure-activated Luer-lock valve 330. The resilient member 225 is hollow and defines a channel that places the injector delivery device conduit 311 in fluid communication with the Luer-lock valve 330. The resilient member 225 is sized such that it fits over the bulb 319, coupling end 322, and second/injection portion 336 and compresses the bulb 319, coupling end 322, and second/injection portion 336 to create a seal that does not allow air or fluid to escape.

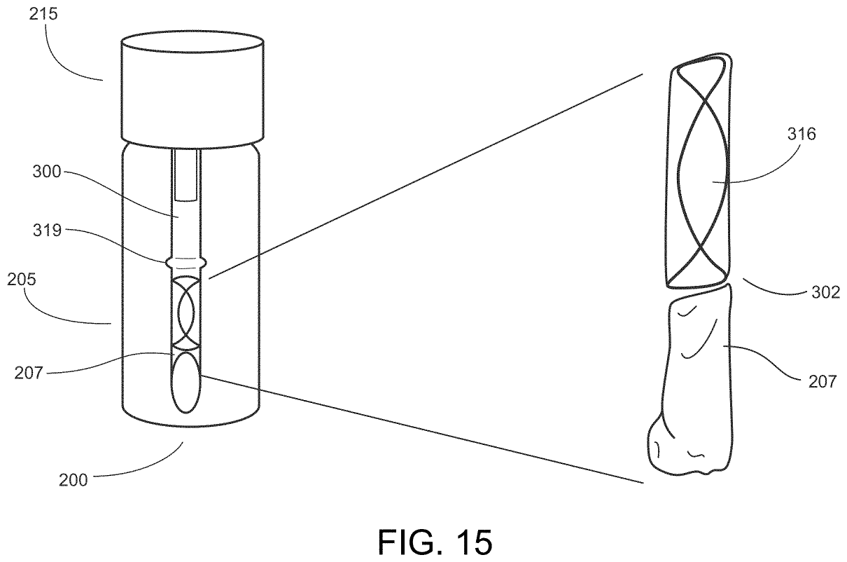

To store and transport the ophthalmic tissue, the injector delivery device 300 can be used in conjunction with an injector carrier 200 as shown in FIG. 15. The injector carrier 200 includes a container 205 having an opening 210, a cap 215 configured to seal the opening 210 of the container 205, and a coupling means configured to connect the coupling end 322 of the injector delivery device 300 to the cap 215. In other embodiments, the coupling means is omitted, and the injector carrier 300 with the resilient member 225 and Luer-Lock valve 330 all connected together are placed in the container 205 as a single unit for storage and transport without being coupled to any component of the injector carrier 200.

The container 205 of the injector carrier 200 holds an amount of corneal storage medium sufficient to contact ophthalmic tissue in the injector delivery device 300 when the injector delivery device 300 is disposed within the container 205 while the cap 215 seals the opening 210 of the container 205. A sponge 207 is inserted into the injection end 302 of the injector delivery device 300 while stored in the container 205 to prevent the ophthalmic tissue for inadvertently escaping the injector delivery device 300 prior to surgery. In other embodiments, a silicon cap is placed over the tip of the injector, rather than a sponge, to prevent the ophthalmic tissue from escaping prior to surgery.

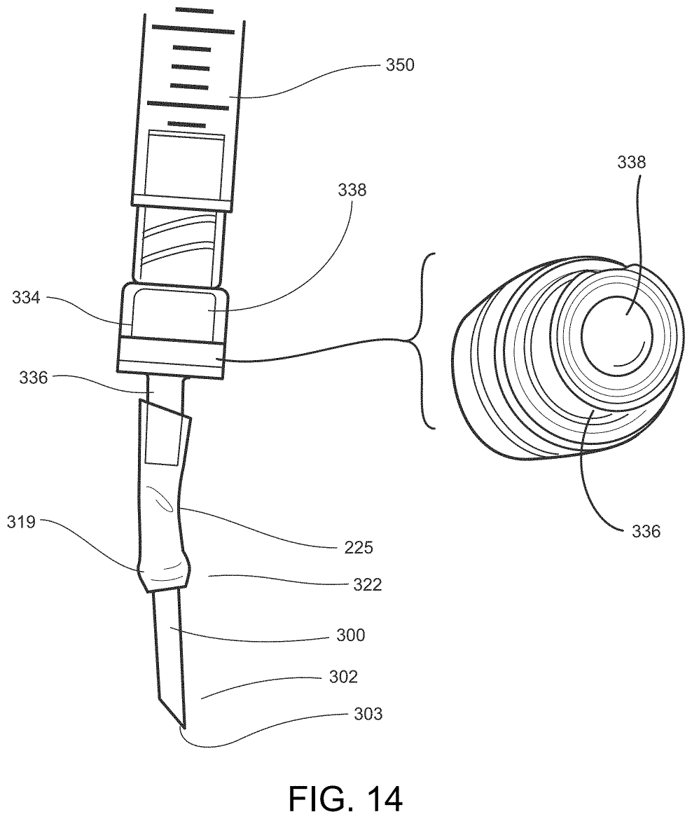

Turning again to FIGS. 13-14, the Luer-lock valve 330 includes a threaded feed portion 332, a main body 334, and a second or injection portion 336. The threaded feed portion 332 and the injection portion 336 both include an opening and a passage that are in fluid communication with the interior of the main body 334. The main body 334 houses a stopper 338 that can be made of silicone or another resilient, compressible material that deforms under pressure but returns to its original form when the pressure is removed. Absent applied pressure, the stopper 338 occupies the entire volume of the interior of the valve main body 334 so that fluid cannot pass from the passage of the threaded feed portion 332 to the passage of the injection portion 336. When pressure is applied to the stopper 338, the stopper 338 deforms and allows fluid to pass from the passage of the threaded feed portion 332 to the passage of the injection portion 336. When the pressure is removed, the stopper 338 returns to its prior shape, thereby cutting off fluid flow.

Those of skill in the art will appreciate that use of a resilient member 225 is not intended to be limiting, and other suitable components and configurations may be used to place a valve 330 in fluid communication with the injector delivery device 300. For example, the injector delivery device 300 may be made of a plastic material with a coupling end 322 opening sized to fit over the injection portion 336 of the Luer-lock valve 330. Alternatively, the coupling end 322 and injection portion 336 may be threaded so that the two components can be screwed together. Additionally, other types of valves may be used instead of the pressure actuated Luer-lock valve 330 to place the syringe 350 in fluid communication with the injector delivery device 300. Other embodiments may utilize a spring-tensioned valve or a butterfly-type valve where applied fluid pressure cause a disk or other stopper to move and allow fluid flow.

With respect to the embodiments shown in the attached figures, the Luer-lock valve 330 facilitates the no-touch feature of DSAEK surgical procedures performed using the injector delivery device 300 described above. Prior to surgery, the threaded feed portion 332 of the Luer-lock valve 330 is connection to a syringe 350 having a barrel and a plunger, as depicted in FIG. 14. The syringe 350 is filled with a balanced salt solution suitable for injection into a patient's anterior chamber. The injection portion 336 of the Luer-lock valve 330 is coupled to the second portion 235 of the resilient member 225, and the first portion 230 of the resilient member 225 is connected to the coupling end 322 of the injector delivery device 330. The injector delivery device conduit 311 houses the ophthalmic tissue to be transplanted and is filled with corneal storage medium such as Optisol-GS to facilitate sterile preservation of the tissue. With the components connected in this manner, the barrel of the syringe 350 is placed in fluid communication with the injector delivery device conduit 311 through the resilient member 225 and the Luer-lock valve 330.

During surgery, the injection end 302 of the injector delivery device 300 is inserted through a pre-cut incision in a patient's cornea, and the injection end 302 is placed inside the anterior chamber with the bevel-side 305 facing “up.” Next, the ophthalmic tissue is injected using fluid into the anterior chamber by actuating the syringe plunger. When the plunger is depressed, the balanced salt solution exerts a positive pressure on the stopper 338. The stopper 338 deforms in response to the positive pressure and allows the balanced salt solution to pass from the syringe 350 barrel through the Luer-lock valve 330 and resilient member 225 to the injector delivery device conduit 311. The balanced salt solution then exerts a positive pressure within the conduit 311 that expels the ophthalmic tissue from the injection end 302 of the injector delivery device 300 into anterior chamber without the need to touch or mechanically contact the ophthalmic tissue. The ophthalmic tissue is expelled from the injector delivery device 300 along with balanced salt solution that flows into the anterior chamber and applies pressure to the ophthalmic tissue to facilitate the tissue unfolding so that the tissue takes the shape of the patient's cornea. The balanced salt solution injected into the anterior chamber also maintains ocular pressure and fluid levels within the anterior chamber.

Eliminating the need for mechanical contacting the ophthalmic tissue has advantages that include, without limitation, reducing damage to the endothelium and allowing transplantation of smaller tissue sizes that would otherwise be challenging to grasp using forceps or other surgical instruments. In some cases, transplanted ophthalmic tissue may have a thickness of 70 microns or less that is impractical to transplant using existing surgical techniques.

In addition to eliminating the need to contact the ophthalmic tissue and endothelium, the above-described procedure has the advantage of dispensing tissue into the anterior chamber along with corneal storage medium or balanced salt solution that assists in tissue unfolding and helps to maintain pressure and fluid levels within the anterior chamber to prevent cornea collapse. As described above, the smaller injector delivery device and incision sizes, the beveled shape of the injector delivery device, and the injected fluid all work in combination to facilitate closed surgical techniques that maintain pressure and fluid levels in the anterior chamber to prevent cornea collapse. As a result of these features, surgical procedures take less time, and patients have improved clinical outcomes, including reduced healing time and fewer surgical complications as compared to conventional surgical techniques.

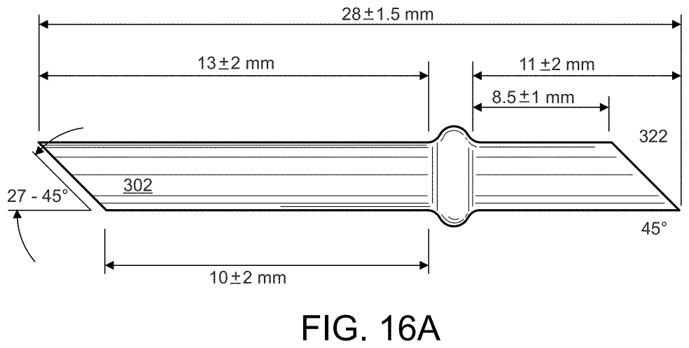

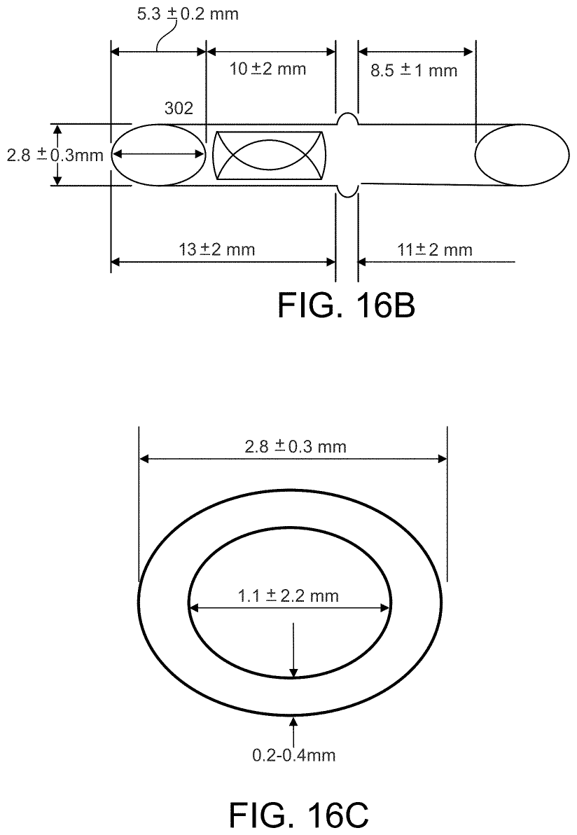

FIGS. 16A, 16B, and 16C illustrate the sizing and dimensions of an example embodiment of the injector delivery device 300 where the injector delivery device 300 has the following dimensions: (i) a total length of 28 mm +/−1.5 mm from the injection end 302 to the coupling end 322; (ii) an outside diameter of 2.8 mm +/−0.3 mm); (iii) an inside diameter of 2.1 mm +/−0.1 mm that defines the size of the conduit 311; (iv) a wall thickness of approximately 0.2 mm to 0.4 mm; (v) an injection end 302 bevel length of 5.3 mm +/−0.2 mm; (vi) a coupling end 322 bevel length of 2.8 mm +/−0.2 mm; (vii) and injection end 302 bevel angle of 27 degrees to 45 degrees; (viii) a couple end 322 bevel angle of 45 degrees +/−2 degrees; and (ix) a bulb 319 diameter of 2.5 mm +/−0.4 mm where the bulb 319 is positioned 13 mm +/−2 mm from the leading cutting edge 303 of the injection end 302.

In one embodiment, the injector delivery device 300 generally has a minimum internal diameter of 1.1 mm, and a minimum external diameter of 2.2 mm to allow safe storage of donor ophthalmic tissue without damaging the tissue while also allowing surgical incision sizes of less than 4 mm. The internal diameter can be increased to a maximum of about 3.2 mm, and the external diameter can be increased up to a maximum of about 4 mm while still allowing surgical incision sizes of 4 mm or less and securing the ophthalmic tissue graft within the conduit 311. The overall length can generally vary from about 27 mm to about 30 mm.

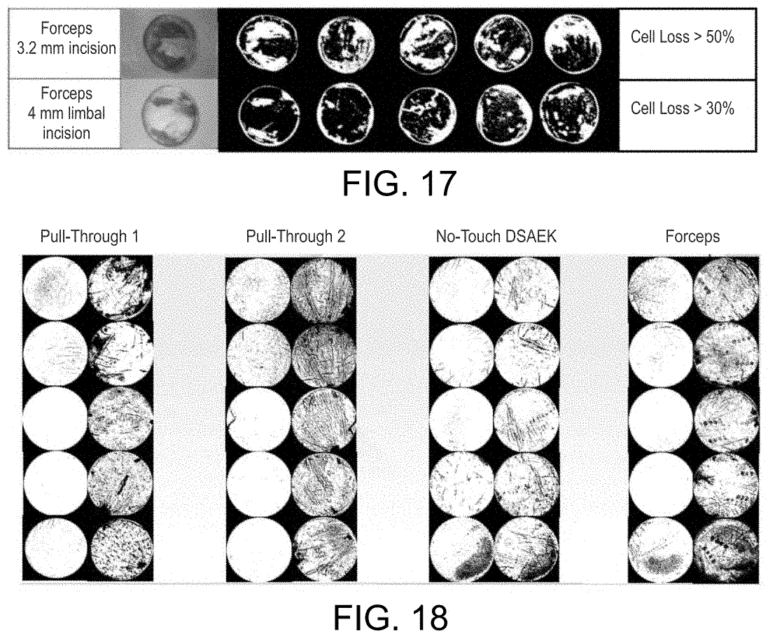

The improvements achieved by the disclosed no-touch DSAEK delivery devices and methods are illustrated with reference to FIGS. 17 and 18 and Table 1 below that definitively show a reduction in endothelial cell loss when using the new touch-free surgical devices and techniques over conventional devices and techniques. More specifically, FIGS. 17 and 18 show stained images of corneal tissue following simulated surgical procedures with the dark regions representing endothelial cell loss. FIG. 17 illustrates endothelial cell loss following forceps-assisted ophthalmic tissue transplant through a 3.2 mm incision and a 4 mm incision and is adapted from the following published article: Effects of a Novel Push-through Technique Using the Implantable Collamer Lens Injector System for Graft Delivery during Endothelial Keratoplasty, Sug Jae Kang, et al., Korean Journal of Ophthalmology, Vol. 27, No. 2 (2013). FIG. 18 and Table 1 illustrate the results of a study commissioned by the present patent applicant to examine endothelial cell loss resulting from use of pull-through surgical techniques, forceps-assisted surgical techniques, and applicant's novel no-touch DSAEK surgical devices and technique.

FIG. 17 shows that traditional forceps-assisted surgical techniques resulted in an endothelial cell loss of greater than 50% for insertion through a 3.2 mm incision and greater than 30% for insertion through a 4 mm incision. Thus, even though smaller incision sizes are more advantageous for the reasons discussed herein (e.g., minimizing fluid loss and astigmatism effects), forceps-assisted surgical techniques are poorly adapted for smaller incision sizes and result in more endothelial cell damage.

With respect to FIG. 17 and Table 1, the forceps-assisted and pull-through surgical techniques all resulted in a mean endothelial cell loss of 22.6% percent or greater. By contrast, the present no-touch surgical devices and techniques showed a mean endothelial cell loss of 7.7%, which represents an approximately two-thirds reduction or improvement in cell loss over pull-through and forceps-assisted techniques. In other words, not only are the disclosed no-touch surgical devices and techniques specially adapted for the use of smaller incision sizes and the concomitant advantages, but the devices and techniques also result in substantially less endothelial cell damage given that the tissue is not subject to mechanical contact with a surgical instrument and pushed or pulled through the corneal incision.

TABLE 1
Endothelial Cell Loss Percentages

| Group | N | Mean | Std. Dev. | Min | Maximum |
|---|---|---|---|---|---|
| Pull-Through 1 | 5 | 22.6 | 4.8 | 18.0 | 30.1 |
| Pull-Through 2 | 5 | 26.9 | 6.4 | 21.0 | 34.8 |
| Forceps | 5 | 23.1 | 8.6 | 16.0 | 37.7 |
| No-Touch DSAEK | 5 | 7.7 | 2.2 | 4.4 | 10.1 |

### Methods for Performing Endothelial Keratoplasty Procedures

Methods for performing endothelial keratoplasty are provided that can employ an fluid injector delivery device and/or an injector carrier as described herein. An injector delivery device shown in FIGS. 1-6 is provided where the first end of the third conduit is fluidly coupled to the second end of the second conduit and ophthalmic tissue is positioned within the second portion of the injector delivery device. The second end of the third conduit can be inserted into the eye of the patient, where a cutting surface cuts and penetrates the eye of the patient. The ophthalmic tissue can then be dispensed from the second conduit of the injector delivery device through the third conduit of the injector delivery device into the eye of the patient.

Other methods for performing endothelial keratoplasty for an eye of a patient are provided that employ variations of the injector delivery device shown in FIGS. 1-6. In one embodiment, the third portion 115 having the cutting surface 175 is removed, and tissue is dispensed through the second portion 110 during surgery. That is, an injector is provided where ophthalmic tissue is positioned within the second portion of the injector delivery device. An incision can be made in the eye of the patient. The second end of the second conduit can be inserted through the incision into the eye of the patient. The ophthalmic tissue can then be dispensed from the second conduit of the injector delivery device into the eye of the patient without the use of the third portion and cutting surface.

In yet another embodiment, an injector delivery device is placed in fluid communication with a syringe through a valve. The injector delivery device houses folded ophthalmic tissue to be transplanted. The syringe is filled with a balanced salt solution or other fluid suitable for injection into the eye of a donor patient. An incision is pre-cut in the patient's eye using a scalpel or other surgical instrument. The injector delivery device is inserted through the incision and occupies most of the incision opening to create a seal that minimizes the amount of fluid that escapes the eye. A plunger on the syringe is depressed to apply positive pressure that opens the valve. Once the valve is opened, fluid flows from the syringe through to the injector delivery device and expels the ophthalmic tissue and an amount of fluid into the eye of the patient. The tissue unfolds once dispensed into the patient's eye, and the unfolding is assisted by fluid being expelled from the injector delivery device.

Methods of storing ophthalmic tissue for use in endothelial keratoplasty are provided that can employ a fluid injector delivery device as described herein and an injector carrier. The injector delivery device can be loaded with the ophthalmic tissue. The loaded injector delivery device can be coupled to the cap of the injector carrier. The loaded injector delivery device can then be disposed into the injector carrier, where the ophthalmic tissue contacts corneal storage medium within the container. It should be noted that such methods can also be practiced using injectors or other delivery devices for endothelial keratoplasty other than the injector delivery device described herein, where the injector carrier is configured to accommodate the other injector or other delivery device. Such methods can further include transporting the injector carrier including the loaded injector delivery device to a site for performing endothelial keratoplasty.

In certain embodiments, the present technology provides various methods of using the injector delivery device. These include ways of loading a graft or ophthalmic tissue into the injector, ways of assembling the injector, and ways of administering the graft or ophthalmic tissue; e.g., performing keratoplasty. The following example methods include a series of steps where it will be evident to one skilled in the art that the order of certain steps can be different in various embodiments while the order of other certain steps cannot be changed relative to each other. Similarly, additional steps can be included in the various embodiments of the present technology and certain steps may be omitted in certain embodiments of the present technology.

The present technology further includes various injector carriers that can be preloaded with ophthalmic tissue and that contain corneal storage medium, where such injector carriers can be used for storage, transport, and transplantation purposes. Various facets from the collection of donor tissue through to the administration of the ophthalmic tissue into a recipient's eye can be improved thereby. For example, benefits are realized for Descemet's membrane endothelial keratoplasty, Pre-Descemet's endothelial keratoplasty, and Descemet's stripping automated endothelial keratoplasty, where at least a portion of the Descemet's membrane, which includes endothelial cells, is removed from a donor cornea and transplanted into a recipient's eye. The layer of the Descemet's membrane can be inserted into the front portion (e.g., anterior chamber) of the recipient's eye using a particular injector delivery device. The present injector carrier can be configured to accommodate various injector delivery devices, including the injector delivery devices described herein, which are preloaded with endothelial tissue grafts, allowing safe and secure storage and transportation of the ophthalmic tissue while in contact with corneal storage medium; e.g., Optisol-GS corneal storage medium. The injector carrier allows easy removal of the injector delivery device from the corneal storage medium for use in endothelial keratoplasty.

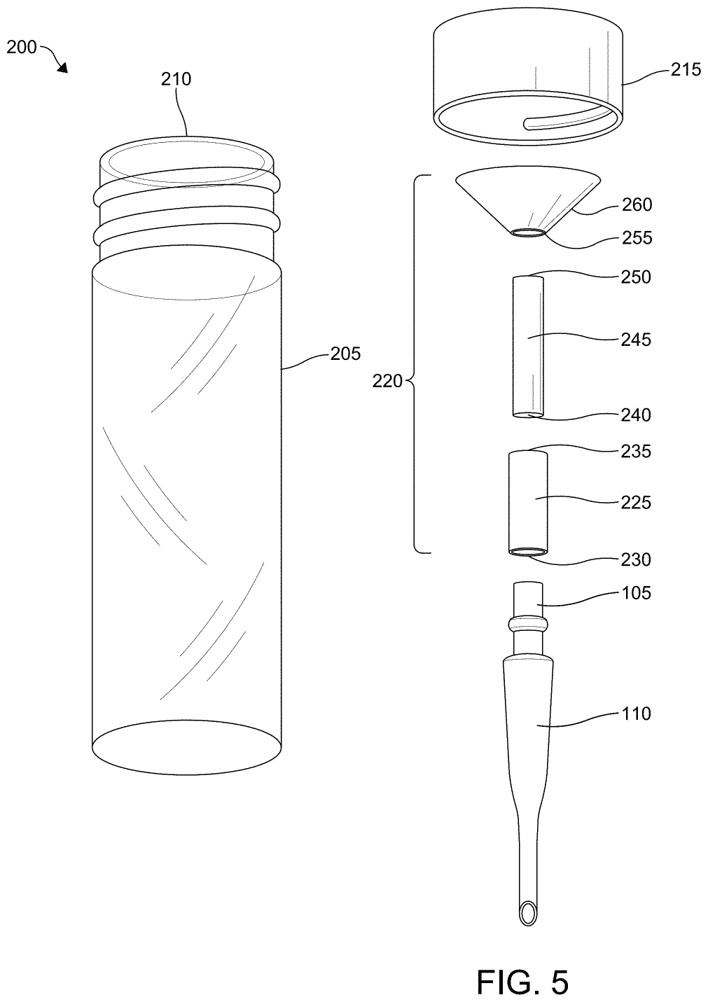

Embodiments of an injector carrier for ophthalmic tissue used in an endothelial keratoplasty can include a container and a lid configured to seal the container. The injector carrier can also include an injector and a coupling means. The injector delivery device can include a conduit fluidly coupling a first end to a second end, where the conduit includes a portion configured to accommodate the ophthalmic tissue for the endothelial keratoplasty. The coupling means 220 shown in FIG. 5 is configured to couple one of the first end and the second end of the injector delivery device to an interior face of the lid. In this way, the injector delivery device depends or projects from the interior face of the lid. Removal of the lid from the container allows easy removal of the injector delivery device, including any ophthalmic tissue loaded into the injector. Corneal storage medium can be provided that contacts the ophthalmic tissue when the ophthalmic tissue is positioned within the injector delivery device.

In certain embodiments, the injector carrier can include a sealing means in the form of a cone that is coupled to the interior face of a cap for a container. The container can take the form of vial having a length greater than its diameter. A stem can act as an intermediate coupling between the cone and a piece of tubing or collar, where the stem has a first end coupled to the cone and a second end coupled to a first end of the tube. A second end of the tube can receive one of the first end and the second end of the injector delivery device. The tubing or collar can be configured to receive one end of the injector delivery device and can be tailored to work with various types, shapes, and configurations of injector delivery devices or delivery devices. For example, the tubing can be resilient and flexible and can slip over an end of the injector delivery device, where depending on the flexible nature, the tubing can stretch and flex to accommodate injector delivery devices having different sized ends. Alternatively, the tubing of the injector carrier can be replaced with a different sized tubing to accommodate a different sized injector end. In other embodiments, the tubing can be deleted and the stem can be configured to be disposed within an end of the injector. The stem can be compressible in such instances. The stem can be configured as a solid stem or plastic cylinder that can be coupled or directly connected securely at an end thereof to the cone on the interior face or inner surface of the cap. The stem can also be a portion of a handle from an ocular sponge used in preparing the ophthalmic tissue graft.

In certain embodiments, the injector delivery device can be made of glass and can connect vertically to the bottom of the stem using the tubing, where the injector delivery device can be disposed within the container (e.g., a glass vial) in a vertical position with minimal handling and movement of the injector delivery device. To remove the injector delivery device from the container, for example, the cap can be a screw cap that needs to be unscrewed, where the cap can be lifted upwards to remove the injector delivery device from the container. The injector delivery device can then be detached from the stem, keeping the tubing connected to the injector delivery device. The tubing can then be connected to a syringe or other device for dispensing the ophthalmic tissue from the injector delivery device as part of an endothelial keratoplasty procedure.

The injector carrier can also be used in various methods of storing ophthalmic tissue. Such methods can include where an injector carrier is provided and ophthalmic tissue is positioned within an injector delivery device. Corneal storage medium is provided within the container of the injector carrier. The corneal storage medium contacts the ophthalmic tissue when the injector delivery device is disposed within the injector carrier. In this way, the injector carrier can maintain viability of the ophthalmic tissue during storage and transport.

The injector carrier can also be used in various methods of transporting ophthalmic tissue. Such methods can include where the ophthalmic tissue is obtained from a donor. An injector carrier as described herein is provided and the ophthalmic tissue is disposed within the injector delivery device. Corneal storage medium is provided within the container, where the corneal storage medium contacts the ophthalmic tissue when the ophthalmic tissue is positioned within the injector delivery device. The injector carrier, including the injector delivery device with the ophthalmic tissue disposed therein, is then transported to a site for the endothelial keratoplasty, for example.

In certain embodiments, the present technology provides various methods of using the injector carrier. These include ways of loading a graft of ophthalmic tissue into the injector delivery device, ways of assembling the injector carrier, and ways of administering the graft or ophthalmic tissue using the injector carrier (e.g., performing keratoplasty). The following exemplary methods include a series of steps where it will be evident to one skilled in the art that the order of certain steps can be different in various embodiments while the order of other certain steps cannot be changed relative to each other. Similarly, additional steps can be included in the various embodiments of the present technology and certain steps may be omitted in certain embodiments of the present technology.

Various embodiments of an injector delivery device and an injector carrier can be used as follows to load ophthalmic tissue (e.g., a prepared graft) as follows.

### Loading a Prepared Graft

1. Use universal scissors to cut a small (approximate ½ inch) piece of suction tubing to fit the injector.

2. Connect one side of the suction tubing to a narrow end of the injector delivery device and the other side to a 1 cc syringe.

3. Depress the plunger of the syringe to transfer media to the injector delivery device from the syringe, ensuring no air bubbles are present.

4. Place a wide end of the injector next to the prepared graft of ophthalmic tissue and use suction from the syringe to move the graft into the injector, until the graft is in a wide central portion of the injector.

5. Gently disconnect the syringe from the tubing at the narrow end of injector, making sure that the wide end of the injector delivery device stays in the corneal storage medium during disconnection.

6. Reconnect syringe to the tubing at the wide end of injector delivery device, making sure that the narrow end of the injector remains submerged in the medium.

7. Plug the narrow end of the injector with a thin (e.g., 1 mm wide) piece of wet eye spear for security; injector is ready for transportation.

8. Couple the tubing to the stem and cone of the coupling means of the injector carrier and place injector in an upright position (narrow end down) into the container (e.g., vial) of the injector carrier, where the container includes corneal storage media.

Various embodiments of the injector carrier can be used (e.g., in an operating room) for administration of the ophthalmic tissue (e.g., a prepared graft) as loaded into the injector as follows.

### Assemble Injector in Operating Room

1. Prepare basin with an intraocular irrigating solution (e.g., balanced salt solution) enough to submerge 3 cc syringe and injector delivery device.

2. Gently remove injector delivery device (containing ophthalmic tissue) from the container of corneal storage media by removing the lid from the container and lifting the lid with attached coupling means (e.g., cone, stem, tubing) and injector delivery device.

3. Place the injector delivery device into the prepared basin with balanced salt solution, decouple the tubing from the stem, leaving the tubing attached to the injector delivery device in the basin, making sure the injector delivery device is submerged and no air bubbles observed inside the injector delivery device.

4A. As submerged, slowly connect the tubing attached at an end of the injector delivery device to valve that is connected to a 3 cc syringe with balanced salt solution and leave it in basin with balanced salt solution.

4B. As submerged, slowly connect the tubing attached at an end of the injector delivery device to a 3 cc syringe with balanced salt solution and leave it in basin with balanced salt solution. That is, as an alternative to step 4A, use of the valve may be omitted in cases where the injector delivery device is configured for connection directly to the syringe.

5. As submerged, gently place the free end of the injector delivery device against the wall of the basin, so the plug touches that wall. Very slowly depress the plunger of the syringe to transfer balanced salt solution from the syringe to the injector delivery device, ensuring there are no air bubbles present. Corneal storage medium will start exiting injector delivery device through the free end and plug and balanced salt solution will replace corneal storage medium inside of the injector delivery device.

6. Leave prepared syringe with connected injector delivery device in the basin.

7. Prepare recipient/patient.

8. As submerged, remove plug from the narrow end of the injector delivery device.

9. Remove the prepared syringe from the basin with connected injector delivery device by holding syringe body and transfer onto operating field.

10. Place narrow end of the injector delivery device next to the periphery of the patient's cornea.

11. By pushing on the syringe body, insert one end of the injector delivery device into the anterior chamber of the recipient through a pre-cut incision or through an incision made using a cutting surface of the injector delivery device, and make sure the end is completely visible in the chamber.

12. Gently depress the plunger of the syringe to transfer prepared ophthalmic tissue graft from the injector into the anterior chamber of the recipient, ensuring there are no air bubbles present.

13. Confirm the presence of the graft in the cavity of the anterior chamber and gently remove narrow end of the injector from the anterior chamber.

14. As needed, perform appropriate steps to unfold donor ophthalmic tissue graft within recipient's eye.

Various benefits and advantages are obtained by the design and use of the injector delivery device provided by the present technology. These include the ability of the injector delivery device to enter into the eye through a smaller incision, which improves patient healing time and reduces complications. In particular, use of a needle injector embodiment described herein does not require a prior blade (e.g., keratome) incision to be entered into eye. This can preserve integrity of the eye and has the potential to decrease astigmatism in the patient receiving the graft. In other embodiments, as smaller injector delivery device is used that is particularly well suited for DSAEK procedures, which also reduces healing time and surgical complications by, among other things, using smaller incision incises and maintaining ocular pressure and fluid levels during transplant surgery.

The injector delivery device also has the advantage that the injector delivery device can flexibly include various configurations. The injector delivery device can be constructed as a unitary component, such as the injector shown in FIGS. 11A-11B. In other embodiments, such as the needle injector shown in FIGS. 1-2, various configurations of the first and second portions along with various types of the third portion can be assembled. For example, one or more third portions can be provided with the needle injector, where the third portions can have various dimensions, bore sizes, bevels, cutting surfaces, etc., where a particular third portion can be selected based on the patient's eye geometry and/or where and how the ophthalmic tissue is to be dispensed.

The injector delivery devices can also be dimensioned to fit within various injector carriers, including various storage/transport containers for holding, storing, and transporting ophthalmic tissue pre-loaded into the injector delivery device. The injector delivery devices described herein having multiple portions can be dimensioned to fit within the injector carrier with or without the third portion of the injector delivery device coupled to the remainder of the injector delivery device. The injector delivery device can be secured in an injector carrier or container or vial along with Optisol-GS corneal storage medium. The injector delivery device is removed from the injector carrier at a site for performing endothelial keratoplasty.

Various benefits and advantages are obtained by the design and use of the injector carrier provided by the present technology. In particular, the coupling means 220 of the injector carrier embodiment shown in FIG. 5 can be configured for coupling with various types of injector delivery devices. Where the coupling means 220 includes the resilient tubing 225, for example, the flexible nature of the tubing can allow various dimensions of injector delivery device ends to be disposed therein. Alternatively, an end of the tubing could be compressed and inserted into an end of an injector delivery device or the tubing can be eliminated and the stem configured to fit within an end of the injector delivery device. The injector carrier can also be provided with multiple coupling means, including one or more cones of various dimensions, one or more stems of various dimensions, and/or one or more tubings of various dimensions to provide a universal set of coupling means so that the injector carrier can accommodate various dimensioned injector delivery devices. For example, the coupling means of the injector carrier can be configured to couple injector delivery devices such as the Striko modified Jones tube, the 1.6 mm LEITR Weiss Glass Cannula, and the 2.8 and 3.0 mm LEITR Weiss Glass Cannula injector delivery device. Notably, the acronym LEITR refers to the current patent applicant, Lions Eye Institute for Transplant and Research.

The injector carrier also reduces the steps and components required for preparation and introduction of donor ophthalmic tissue, as where the tubing of the injector carrier can also function as a suction tube for loading the injector with the ophthalmic tissue and as a coupling interface with a syringe for dispensing the ophthalmic tissue from the injector delivery device. The stem can also be derived from the handle of an ocular sponge used during preparation of the ophthalmic tissue graft. The injector carrier also minimizes the chance that the ophthalmic tissue will stick to an inner surface of the injector, where the injector carrier allows vertical positioning of the ophthalmic tissue during storage and/or transportation. The injector carrier further improves removal of the injector from the container (e.g., storage/transport vial), where the coupling of the injector delivery device to the inner surface of the lid allows simple removal of the lid from the container and withdrawal of the injector from the container using the lid (e.g., where the lid is unscrewed from the vial and lifted upwards to remove the injector delivery device from the container containing corneal storage medium). The tubing of the coupling means can also be removed along with the injector delivery device from the remainder of the injector carrier, where the tubing, still attached to the injector delivery device, can be coupled to a syringe or other device for dispensing the ophthalmic tissue from the injector during endothelial keratoplasty, for example. The tubing can also be flexible to universally accommodate the coupling of syringes of various dimensions and configurations.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. An ophthalmic tissue delivery apparatus for performing endothelial keratoplasty comprising:
(a) an injector that comprises
(i) an elongated hollow body,
(ii) an inner surface that defines a conduit,
(iii) a beveled first end having a first opening, and
(iv) a second end having a second opening;
(b) a pressure actuated valve that comprises
(i) a first portion configured for coupling to a syringe,
(ii) a valve main body,
(iii) a deformable stopper disposed in the main body, and
(iv) a second portion in fluid communication with the injector second end.

2. The ophthalmic tissue delivery apparatus of claim 1, wherein:
(a) the conduit comprises an inner diameter between 1.1 millimeters and 3.2 millimeters;
(b) the injector comprises an outer diameter between 2.2 millimeters and 4.0 millimeters; and
(c) the beveled first end has a bevel outer diameter that less is than 4 millimeters.

3. The ophthalmic tissue delivery apparatus of claim 1 further comprising:
(a) an elongated resilient member having a channel extending from a first aperture to a second aperture, wherein
(i) the resilient member is coupled to the second portion of the pressure-actuated valve by extending the second portion partially through the resilient member first aperture, and wherein
(ii) the resilient member is coupled to the injector second end by extending the injector second end partially through the resilient member second aperture.

4. The ophthalmic tissue delivery apparatus of claim 1 further comprising a syringe filled with a balanced salt solution coupled to the first portion of the pressure actuated value.

5. The ophthalmic tissue delivery apparatus of claim 1, wherein the elongated hollow body further comprises a bulb disposed on an outer surface of the elongated hollow body.

6. An ophthalmic tissue apparatus for performing endothelial keratoplasty comprising:
(a) an elongated hollow body;
(b) an inner surface of the elongated hollow body, wherein
(i) the inner surface defines a conduit, and wherein
(ii) the conduit comprises an inner diameter between 1.1 millimeters and 3.2 millimeters;
(c) an outer diameter of the elongated hollow body, wherein the outer diameter is less than 4 millimeters;
(d) a beveled first end having a first opening;
(e) a second end having a second opening, wherein the second end is coupled to, and in fluid communication with, a fluid manipulation device.

7. The ophthalmic tissue delivery apparatus according to claim 6 further comprising an injector carrier, wherein the injector carrier comprises:
(a) a container having an opening;
(b) a cap configured to seal the opening of the container; and
(c) at least a portion of the injector is disposed within the container.

8. The ophthalmic tissue delivery apparatus according to claim 7, wherein the conduit and the container are each at least partially filled with a corneal storage medium.

9. The ophthalmic tissue delivery apparatus according to claim 8, wherein
(a) the conduit houses an ophthalmic tissue graft comprising an endothelium layer and a stromal layer, and the ophthalmic tissue graft is in a folded configuration with the endothelium layer facing inward and the stromal layer facing outward to contact the conduit inner surface; and
(b) the ophthalmic tissue graft endothelium layer is in contact with the corneal storage medium.

10. The ophthalmic tissue delivery apparatus according to claim 6, wherein:
(a) the beveled first end comprises a leading portion and a trailing portion;
(b) the leading portion comprises a cutting surface for cutting and penetrating eye tissue; and
(c) the trailing portion comprises a non-cutting surface.

11. The ophthalmic tissue delivery apparatus according to claim 6 further comprising a pressure actuated valve coupled to the injector second end.

12. The ophthalmic tissue delivery apparatus according to claim 11 further comprising a syringe coupled to the pressure actuated value.

13. The ophthalmic tissue delivery apparatus according to claim 11, wherein the pressure actuated value comprises:
(a) a first portion configured for coupling to a syringe;
(b) a valve main body;
(c) a deformable stopper disposed in the main body; and
(d) a second portion coupled to the injector second end.

14. The ophthalmic tissue delivery apparatus according to claim 12, wherein:
(a) the pressure actuated value is coupled to the injector second end through a resilient member; and wherein
(b) the resilient member comprises a channel extending from a first aperture to a second aperture; and wherein:
(c) the resilient member is coupled to the second portion of the pressure-actuated valve by extending the second portion partially through the resilient member first aperture; and wherein
(d) the resilient member is coupled to the injector second end by extending the injector second end partially through the resilient member second aperture.

15. The ophthalmic tissue delivery apparatus according to claim 12, wherein:
(a) the elongated hollow body further comprises a bulb disposed on an outer surface of the elongated hollow body; and
(b) the injector second end extends through the resilient member second aperture such that bulb is within the resilient member channel.

16. A method for storing ophthalmic tissue comprising:
(a) providing the ophthalmic tissue graft delivery apparatus of claim 8;
(b) loading the conduit with an ophthalmic tissue graft, wherein the ophthalmic tissue graft contacts the corneal storage medium; and
(c) transporting the injector and carrier to a site for performing endothelial keratoplasty.

17. A method for performing endothelial keratoplasty for an eye of a patient comprising:
(a) providing an ophthalmic tissue graft delivery apparatus according to claim 1;
(b) loading the conduit with a corneal storage medium;
(c) loading the conduit with an ophthalmic tissue graft, wherein
(i) the ophthalmic tissue graft comprises an endothelium layer and a stromal layer opposite the endothelium layer, and wherein
(ii) the ophthalmic tissue graft is folded with the endothelium layer facing inward and the stromal layer facing outward to contact the conduit inner surface;
(d) coupling a syringe to the first portion of the pressure actuated valve, wherein the syringe comprises (i) a barrel containing a fluid, and (ii) a plunger partially housed within the barrel such that depressing the plunger applies pressure to the fluid;
(e) creating an incision in the eye of a patient, wherein the incision is less than 4 millimeters in length;
(f) inserting the beveled first end into the eye of a patient through the incision, wherein
(i) the beveled first end occupies substantially the entire incision, and wherein
(ii) the bevel faces upward in a direction away from the interior of the patient's eye; and
(g) depressing the plunger to dispense the ophthalmic tissue graft from the conduit through the beveled first end into the eye of the patient.

18. The method for performing endothelial keratoplasty of claim 17, wherein depressing the plunger causes the fluid to flow from the barrel, through the pressure actuated valve and the injector, and into the patient's eye to exert a positive pressure within the eye.

19. The method for performing endothelial keratoplasty of claim 17, wherein the fluid is a balanced salt solution.
